(19) United States
(12) Patent Application Publication
(10) Pub. No.: US 2026/0151184 A1
(43) Pub. Date: Jun. 4, 2026

(54) SYSTEM AND METHOD FOR VERTEBRAL LOCATION PREDICATION IN SPINE SURGERY

(71) Applicant: Raymond Weihau Hwang, Sudbury, MA (US)

(72) Inventor: Raymond Weihau Hwang, Sudbury, MA (US)

(21) Appl. No.: 18/740,947

(22) Filed: Jun. 12, 2024

Related U.S. Application Data
(60) Provisional application No. 63/551,388, filed on Feb. 8, 2024.

(51) Int. Cl. A61B 34/10; A61B 34/20; A61B 34/32; G16H 10/60

(52) U.S. Cl. CPC A61B 34/10; A61B 34/20; A61B 34/32; G16H 10/60; A61B 2034/104; A61B 2034/105; A61B 2034/2065

(57) ABSTRACT

A system and method for predicting spinal motion and vertebral locations and movement in surgeries through training and using predictive models generated using biomedical profiles of patient spines and surgical patient-specific features. A trained predictive model is used in combination with patient-specific features associated with a surgical patient such that during a real-time surgery the patient-specific features are provided to the trained predictive model and a segmental motion model is created. Using this real-time surgical data and interoperative data, the trained predictive model is updated which then used for updating the segmental motion model such that the updated segmental motion model may be used for producing a series of vertebral location predictions specific to movements and location of the spine of the surgical patient that may be provided to a surgical guidance system for use in performing the real-time surgery in accordance with a surgical plan.

### CROSS-REFERENCE TO RELATED APPLICATION

This application claims the benefit of U.S. Provisional Patent Application No. 63/551,388 filed Feb. 8, 2024, which is hereby incorporated by reference herein in its entirety,

### FIELD OF THE INVENTION

The present invention relates generally to surgical navigation systems, and more particularly, to a system and method employing artificial intelligence for predicting vertebral motion and locations of a patient's spine for use by a surgical navigation or robotic system in real time during surgery.

### BACKGROUND OF THE INVENTION

Computer-assisted surgery technology has become a major development focus in surgery. This technology extends the limited vision of surgeons and enables new surgical techniques and approaches. The surgical navigation system, that is, a surgical system that uses computer-assisted technology, including robotics, has now been used in urology, spine surgery, thoracic surgery, joint replacement surgery and otolaryngology, among others. This technology can track the patient and surgical instruments in real-time, just like navigating airplanes or ships, so it is aptly named a navigation system. Applying such technology has led to improving the accuracy of surgical positioning, reducing surgical trauma and incision sizes, shortening operation duration, and improving surgical outcomes.

Current surgical navigation systems generally adopt the following principle: a surgeon holds a surgical instrument with a traceable mark to perform an operation on the surgical target site (e.g., the patient's spine), also with a traceable mark. The special positioning and aiming operation of the surgical instrument are monitored by a tracer connected to a computer, which simultaneously tracks the surgical target site. The navigation system calculates the relative relationship of the instrument and the surgical site. This relationship is displayed for the surgeon on preoperative or intraoperative multi-mode images, so as to guide the surgeon to operate the surgical instrument and implement the corresponding surgical operation. Thus, a virtual operation environment is executed through digital medical images for surgeons that provides visual support to make surgical operations more accurate, less invasive, and safer. The surgical process generally follows a process such as: (i) obtain patient preoperative imaging (e.g., cat scan (CT)/X-rays images); (ii) import these images into a computer system to perform necessary processing, such as noise reduction and three-dimensional (3D) reconstruction; (iii) perform intraoperative registration, where spatial matching (i.e., registration) is based on intraoperative images, a positioning tracer, and preoperative images, to obtain a spatial position relationship between the surgical instrument and the patient's surgical site; (iv) establish a simulation model in the monitoring computer to display the position of surgical instrument in real-time; and (v) perform the operation, track the surgical instruments and the surgical site, and guide the operation according to the preoperative planning.

For example, surgical procedures on the spine are generally complex procedures involving many steps. These surgeries are necessary given that degenerative disease of the spine is a common cause of disability. Spinal pathology such as spondylolisthesis, scoliosis, and stenosis can lead to severe pain and loss of function and spinal surgery such as fusion can significantly improve pain, function, and spinal alignment in the setting of these conditions. Generally speaking, contemporary spine surgery involves decompressing and/or fusing one or more segments of the spine. Decompression involves removal of bone, ligament, and/or disc material to relieve pressure on the neural elements. Fusion typically utilizes implants (e.g., screws, rods and/or spacers/cages) to impart stability to the spine and encourage bone growth across the intended spinal segments. Accurate decompression and placement of these implants is paramount for safe and efficacious surgery. Overly aggressive decompression can lead to instability while insufficient decompression can lead to persistent symptoms due to residual nerve compression. Misplaced implants can negatively affect critical structures such as nerves, dural sac and blood vessels, and may substantially reduce bone purchase, which may lead to implant loosening or breakage and failure of the fusion.

A number of technologies have been deployed to help assist the surgeon in the accurate placement of spinal implants. However, anatomic variation across patients is common thereby introducing operational variables that need to be accounted for. Therefore, enabling technologies are routinely utilized in contemporary spine surgery. Intra-operative image guidance with fluoroscopy was commonly used but had the limitation of providing only two-dimensional information about a complex three-dimensional structure. Stereotactic navigation allowed for the tracking of surgical instruments on three-dimensional representations of the patient's spinal anatomy. More recently, robotic guidance has combined stereotactic navigation with a guidance arm.

Stereotactic navigation relies on the registration of the patient on the operating table to an imaging study, such as a computer tomography scan, using a registration device, which is typically on or attached to the patient at one point on the spine or pelvis. The assumption is that knowing the position of that registration device allows the navigation system to know the position of the rest of the spine. However, the spine is a flexible structure. The individual vertebral positions can change relative to the reference device for various reasons. These include relaxation over time (e.g., either from medications related to anesthesia or from gravity), deflection from pressure applied to the spine during surgery, removal of bone, intentional modification of spinal alignment (e.g., with a cage/spacer) or some combination of these factors. These changes in spine position are not detected by the navigation system and thus can lead to errors in tracking. This is the so-called “segmental tracking” problem in spine surgery. While attaching trackers to every vertebra theoretically could provide positional information to overcome this, this is both not currently feasible with the optical technology currently in use (i.e., limited tracking geometries limit the number of objects that can be tracked) nor is it practical (e.g., this crowds the surgical field and obstructs the surgery).

Accordingly, there is a need for addressing the segmental tracking problem for improving the capabilities of surgical navigation systems and/or surgical robotic systems.

### SUMMARY OF THE INVENTION

The present invention is directed to providing a system and method for predicting spinal motion and vertebral locations and movement in real-time surgeries using predictive models generated using biomedical profiles of patient spines and surgical patient-specific features.

In a first implementation of the invention, a vertebral location prediction system facilitates predicting spinal motion and vertebral locations and movement in real-time surgeries using predictive models generated using biomedical profiles of patient spines and surgical patient-specific features. The vertebral location prediction system comprising at least: a processor, and a memory storing instructions that when executed cause the processor to perform operations comprising: receiving a trained predictive model; receiving a plurality of patient-specific features associated with a particular one surgical patient; and during a real-time surgery involving the particular one surgical patient:
(i) providing the plurality of patient-specific features received to the trained predictive model and using at least the trained predictive model, creating a segmental motion model specific to the particular one surgical patient;
(ii) receiving real-time surgical data specific to the particular one surgical patient;
(iii) receiving real-time intraoperative data comprising one or more of elapsed surgical time, anesthesia administered, patient position, surgical instrument positioning data, imparted spinal force data, intraoperative imaging, and surgical interventions;
(iv) updating the trained predictive model using at least the real-time surgical data specific to the particular one surgical patient received and the real-time interoperative data received and using the trained predictive model updated, updating the segmental motion model created specific to the particular one surgical patient;
(v) applying the segmental motion model updated by at least inputting the real-time surgical data collected into the segmental motion model updated and receiving from the segmental motion model updated a series of vertebral location predictions specific to movements and location of a spine of the particular one surgical patient;
(vi) providing to a surgical guidance system at least the series of vertebral location predictions received for use in performing the real-time surgery; and
(vii) repeating steps (ii) through (vi) for a defined period during the real-time surgery.

In a second aspect, a method is provided for predicting spinal motion and vertebral locations and movement in real-time surgeries using predictive models generated using biomedical profiles of patient spines and surgical patient-specific features. The method comprising: (a) receiving a trained predictive model; (b) receiving a plurality of patient-specific features associated with a particular one surgical patient; and (c) during a real-time surgery involving the particular one surgical patient: (i) providing the plurality of patient-specific features received to the trained predictive model and using at least the trained predictive model, creating a segmental motion model specific to the particular one surgical patient; (ii) receiving real-time surgical data specific to the particular one surgical patient; (iii) receiving real-time intraoperative data comprising one or more of elapsed surgical time, anesthesia administered, patient position, surgical instrument positioning data, imparted spinal force data, intraoperative imaging, and surgical intervention; (iv) updating the trained predictive model using at least the real-time surgical data specific to the particular one surgical patient received and the real-time interoperative data received and using the trained predictive model updated, updating the segmental motion model created specific to the particular one surgical patient; (v) applying the segmental motion model updated by at least inputting the real-time surgical data collected into the segmental motion model updated and receiving from the segmental motion model updated a series of vertebral location predictions specific to movements and location of a spine of the particular one surgical patient; (vi) providing to a surgical guidance system at least the series of vertebral location predictions received for use in performing the real-time surgery, and (vii) repeating steps (ii) through (vi) for a defined period during the real-time surgery.

In a third aspect, a vertebral location prediction application (alternatively referred to herein as an “app”) may be executed on the vertebral location prediction system and/or another data processing system for executing at least the method operations for predicting spinal motion and vertebral locations and movement in real-time surgeries using predictive models generated using biomedical profiles of patient spines and surgical patient-specific features.

In a fourth aspect, a method is provided for training a predictive model useful in predicting spinal motion and vertebral locations and movement using biomedical profiles of patient spines and surgical patient-specific features. The method comprising: (i) receiving biomedical profiles of the spine associated with multiple patients; (ii) creating a training dataset using the biomedical profiles received and patient factors specific to each patient associated with a respective biomedical profile; (iii) creating a first subset of training data from the training dataset created; (iv) training a predictive model for vertebral location predictions using the first subset of data from the training dataset created; (v) creating a second subset of training data from the training dataset created, the second subset of training data being different from the first subset of training data; and (vi) validating the predictive model trained using the second subset of training data created.

In another aspect, training a predictive model using a first training dataset comprising a plurality of biomedical spine profiles wherein each respective biomedical spine profile is associated with a respective one patient of a plurality of patients and a plurality of patient factors specific to the respective one patient.

In another aspect, the training dataset is updated with the particular one surgical patient's biomedical spine profile.

In another aspect, the surgical guidance system is a surgical navigation system.

In another aspect, the surgical guidance system is a surgical robotic system.

In another aspect, the surgical guidance system comprises both a surgical navigation system and a surgical robotic system.

In another aspect, each biomedical spine profile comprises imaging data and biomechanical data.

In another aspect, each biomedical spine profile further comprises at least one of biomarker data, demographic data, and anthropomorphic data.

In another aspect, the imaging data further comprises at least one or more spine images; the biomarker data further comprises at least one of inflammation data, bone turnover data, cartilage turnover data, and bone metabolism data or other relevant biomarker data; the demographic data further comprises at least one of age, sex, race, height, and weight; the anthropomorphic data further comprises one or more measurements of a body and an associated spine defined by at least one of vertebral body morphology and geometry, spinopelvic parameters, spinal alignment, bone quality, intervertebral disc height and geometry, musculature parameters, ligament measurements, and body composition; and the biomechanical data further comprises at least quantitative dynamic or static measurements of spine loading and/or motion.

In another aspect, there is receiving a plurality of volumetric spine images; defining a second training dataset from the plurality of volumetric spine images; training a machine learning algorithm using the second training dataset defined; and using the machine learning algorithm trained, extracting the anthropomorphic data from the plurality of volumetric spine images received.

In another aspect, the series of vertebral location predictions specific to movements and location of a spine of the particular one surgical patient are continually updated throughout the real-time surgery.

In another aspect, the training of the predictive model employs at least one image-based artificial intelligence (AI) algorithm.

In another aspect, a communications link with one or more websites.

In another aspect, the at least one image-based artificial intelligence algorithm is applied, such as a convolutional neural network (CNN) algorithm.

In another aspect, at least one statistical learning algorithm is applied to at least the biomechanical data and the biomarker data and identifying one or more relationships therebetween that influence spine motion.

In another aspect, a surgical plan associated with the real-time surgery is updated based on at least the series of vertebral location predictions received.

In another aspect, a surgical plan includes at least performing bone removal from the spine of the particular one surgical patient or placement of one or more implants in the spine of the particular one surgical patient.

The methods and systems described herein can be implemented by data processing systems, such as one or more smartphones, tablet computers, desktop computers, laptop computers, smart watches, wearable, audio accessories, on-board computer, and other user devices and consumer electronic devices. The methods and systems described herein can also be implemented by one or more data processing systems which execute executable computer program instructions, stored in one or more non-transitory machine readable media that cause the one or more data processing systems to perform the one or more methods described herein when the program instructions are executed. Thus, the embodiments described herein can include methods, data processing systems, and non-transitory machine readable media.

The above summary does not include an exhaustive list of all embodiments in this disclosure. All systems and methods can be practiced from all suitable combinations of the various aspects and embodiments summarized above, and also those disclosed in the detailed description below.

These and other objects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 presents a flowchart of illustrative operations for predicting spinal motion and vertebral locations and movement in real-time surgeries using predictive models generated using biomedical profiles of patient spines and surgical patient-specific features in accordance with an embodiment;

FIG. 2 presents a flowchart of illustrative operations for training a predictive model useful in predicting spinal motion and vertebral locations and movement using biomedical profiles of patient spines and surgical patient-specific features in accordance with an embodiment;

FIG. 3 presents a diagram of an illustrative surgical operation room in which a vertebral location prediction system, a surgical guidance system and other surgical equipment are disposed around a surgical patient and surgical personnel in accordance with an embodiment;

FIG. 4 presents a diagram of an illustrative real-time surgical operation in which a vertebral location prediction system, a surgical guidance system and other surgical equipment are in use and disposed around a surgical patient and surgical personnel in accordance with an embodiment;

FIG. 5 presents an illustrative anatomical example based on predictive modelling and a comparison of anatomy and location thereof based on predictive modelling in accordance with an embodiment and without the use of such predictive modelling;

FIG. 6 presents an illustrative navigated surgery workflow using a vertebral location prediction system configured in accordance with an embodiment;

FIG. 7 presents an illustrative a vertebral location prediction system configured for predicting spinal motion and vertebral locations and movement in real-time surgeries using predictive models generated using biomedical profiles of patient spines and surgical patient-specific features in accordance with an embodiment; and FIG. 8 presents an illustrative architecture for vertebral location prediction app in accordance with an embodiment.

Like reference numerals refer to like parts throughout the several views of the drawings.

### DETAILED DESCRIPTION

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Shown throughout the figures, the present invention is directed toward a system and method for predicting spinal motion and vertebral locations and movement in real-time surgeries using predictive models generated using biomedical profiles of patient spines and surgical patient-specific features. That is, in accordance with the principles of the disclosed embodiments, a trained predictive model is used in combination with a plurality of patient-specific features associated with a surgical patient such that during a real-time surgery involving the surgical patient the plurality of patient-specific features are provided to the trained predictive model. Then, using at least the trained predictive model, a segmental motion model is created specific to the surgical patient. Real-time surgical data specific to the surgical patient is received as well as real-time intraoperative data comprising one or more of elapsed surgical time, anesthesia administered, patient position, surgical instrument positioning data, imparted spinal force data, intraoperative imaging and surgical interventions. Using this real-time surgical data and interoperative data, the trained predictive model is updated which is then used for updating the segmental motion model created specific to the surgical patient. In this way, the updated segmental motion model updated may be used for producing a series of vertebral location predictions (which may be continuously updated for a defined period of the real-time surgery or throughout the entirety thereof) specific to movements and location of a spine of the surgical patient that may be provided to a surgical guidance system for use in performing the real-time surgery in accordance with a surgical plan. In this way, the surgical guidance system and associated methodology addresses segmental tracking problems for improving the capabilities of surgical navigation systems and/or surgical robotic systems. As a result, the vertebral location prediction system and method of the disclosed embodiments provide an advantageous improvement of practical applications that include surgical guidance systems, orthopedic surgeries and associated surgical procedures, and predictive modelling.

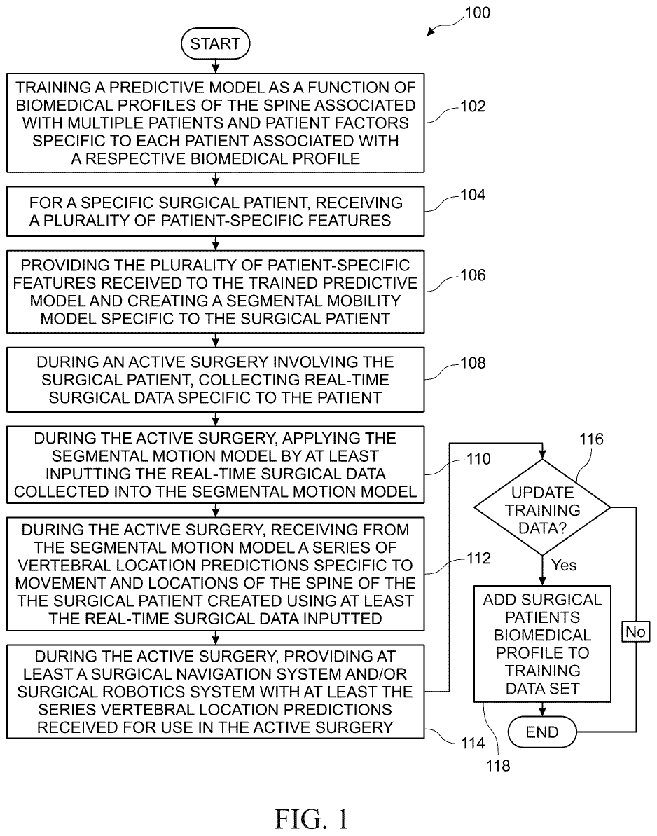

Turning our attention to FIG. 1, a flowchart of illustrative operations 100 is shown for predicting spinal motion and vertebral locations and movement in real-time surgeries using predictive models generated using biomedical profiles of patient spines and surgical patient-specific features in accordance with an embodiment. More particularly, at step 102, training a predictive model using a training dataset comprising a plurality of biomedical spine profiles wherein each respective biomedical spine profile is associated with a respective one patient of a plurality of patients and a plurality of patient factors specific to the respective one patient. Of course, in a further embodiment, the trained predictive model may be received in advance and/or stored in a database for retrieval. In accordance with the principles of the disclosed embodiments, the trained predictive model will facilitate spinal segment tracking without directed monitoring (i.e., attachment, visualization, etc.) of the spinal segments in question. The predictive model is developed by training on at least two (2) primary datasets: biomechanical profiles of the spine as determined by, for example, dynamic imaging and/or cadaveric studies, and various patient factors including, but not limited to, biomarkers, demographics, and comorbidities associated with those patients that affect such profiles. Advantageously, training the predictive model in this manner will enhance the identification of relevant features and how such features affect spinal mechanics (e.g., greater age is associated with stiffer spines and less segmental motion). Further, advantageously, the predictive model may be then deployed during navigated and/or robotic surgery to provide segmental localization of the patient's spine for use by the surgeon, for example, performing the surgery. To accomplish this, the predictive model employed various inputs from the surgical field to provide surgical patient-specific predictions of vertebral positions. These inputs may include the location of surgical instruments, surgical interventions delivered (e.g., laminectomy), intraoperative imaging and forces/pressures applied to the spine. These real-time predictions are then provided to the surgical guidance system thereby providing an update to the expected vertebral positions such that the surgeon may then see the corrected position of the segments (e.g., on a visual display).

Before continuing with the operational aspects of FIG. 1, a more detailed discussion will now proceed with respect to the array of patient factors and spinal mechanics associated therewith that, as noted above, are important to the training of the predictive model. In accordance with the principles of the disclosed embodiments the training dataset may include the following input data:
Imaging data: Spine imaging including magnetic resonance imaging (magnetic resonance imaging (MRIs); upright and supine), CT scans, and X-rays (static and dynamic). These may be preprocessed into standardized cropped images focused on the spine region.
Biomarker data: Quantitative biochemical markers related to inflammation (e.g. C-reactive protein (CRP), Interleukin-6 (IL-6)), bone turnover (e.g., alkaline phosphatase (ALP)), twin reversed arterial perfusion (TRAP)), cartilage turnover (e.g., Coll2-1, Col12-INO2, CS846), bone metabolism (e.g., vitamin D, parathyroid hormone (PTH)) and others that may influence spine mechanics. These would be normalized continuous variables.
Demographic data: For example, age, sex, race, height, weight. Encoded into normalized continuous variables or categorical binary variables.
Anthropomorphic data: Measurements of the body and spine such as vertebral body shape/geometry, spinopelvic parameters, spinal alignment, bone quality, intervertebral disc height and geometry, musculatures parameters (e.g., cross-sectional area, and density) ligament measurements (e.g., thickness, and other dimensions) and body composition (e.g., lean muscle mass, body fat percentage, and torso shape). Normalized continuous variables.
Biomechanical data: Quantitative dynamic or static measurements of spine loading or motion. Normalized continuous variables.

For training, in an embodiment, the imaging and biomechanical data are mandatory elements of each patient biomedical profile given that prior knowledge of what the spine looks like and how it moves is essential. In other embodiments, one or more of the other data elements may be included to provide more information and add richness to the predictive capability of the machine learning algorithm and account for the impact of these additional factors. Any given patient's biomedical profile can have none, some or all of these additional elements. All of the above elements, other than the anthropomorphic data, can be directly input for algorithm training of the predictive model. The anthropomorphic data requires a second machine learning algorithm to be trained in order to extract these values from the imaging data automatically. More particularly, in a preferred embodiment, the machine learning architecture to extract the anthropomorphic features could be defined as follows:

### 1. Spine Segmentation

3D U-Net Convolutional Neural Network (CNN) segments spinal images into relevant anatomy such as vertebral bodies, intervertebral discs, spinal canal, ligaments, facet joints, etc.

### 2. Landmark Detection

Residual Convolutional Neural Network detects anatomical landmarks on vertebral corners, endplates, and spinous processes

### 3. Measurement Regression

Bounding boxes for each vertebral body are extracted
Cropped vertebral body images are input to a CNN
CNN regresses measurement values:
   Vertebral body heights (anterior, central, posterior)
   Spinopelvic parameters
   Spinal curvatures (lordosis/kyphosis angles)
   Intervertebral disc height
   Spinal canal dimensions

### 4. Bone Density Regression

Hounsfield units for trabecular vertebral bodies extracted
Feedforward neural network (FNN) predicts bone mineral density

### 5. Geometry-Based Calculations

Ligament Dimensions:
   Points are sampled along the length of the segmentations of the anterior longitudinal ligament, posterior longitudinal ligament, ligamentum flavum, interspinous ligament, and supraspinous ligament
   The 3D distance between sampled points defines the thickness and cross-sectional area at each region along the entire length of the respective spinal ligaments
Intervertebral Disc Geometry:
   The disc height, disc bulge, and total disc volume are calculated from the segmented intervertebral discs:
      Disc height measured directly as superior endplate to inferior endplate distance
      Disc bulge diameter measured from projected disc area in anterior-posterior direction
      Total volume from voxel summation in the disc's 3D segmentation
   Additionally, quantitative MRI maps like T1rho and T2 relaxation times for disc compartments can indicate proteoglycan and collagen contents to assess mechanical properties and degeneration
Muscle Cross Sectional Area:
   Axial spinal slices are extracted oriented perpendicular to the spine curvature
   Manual or automatic muscle group segmentation may be available in some imaging datasets
   If unavailable, specific back muscle regions can be approximated through geometric rules relative to the segmented vertebral landmarks
   Total enclosed cross-sectional area measured for muscles like the erector spinae or multifidus at each spinal level.
      Thus, the training data would consist of volumetric spine images with manually defined anatomical landmarks and ground truth anthropomorphic measurement values. In this way, a variety of well understood supervised regression loss functions can train the parameters of the neural networks to output accurate anthropomorphic measurements from the input images in an automated manner. Of course, this is only one example of the machine learning/artificial intelligence methods that can be used to achieve an outcome in accordance with the principles of the disclosed embodiments As will be readily appreciated by those skilled in the art, other techniques may also be used to achieve the same outcome in terms of computational speed or with less computational expenditure.

As noted above, in addition to the above-detailed training data provided to the predictive model there is also surgical field data provided. These additional inputs may include surgery time elapsed (e.g., to account for relaxation of the spine over time), positioning (e.g., prone, pad placement), forces imparted upon the spine (e.g., whether within the surgical field or otherwise, to account for instantaneous and aggregate displacement), surgical interventions such as bone removal or implant placement (e.g., to account for intentional changes to anatomy), and surgical instrument position (e.g., to account for the location and direction of forces imparted on the spine). Some examples of the foregoing surgical data include, but are not limited to:
Surgery time elapsed—A clock internal to the system can track the duration of the case and duration of forces to the spine to account for relaxation of the spine as well as any cumulative displacement of the spine over time.
Positioning—The system captures, through surgeon input, the orientation of the patient (e.g., prone, lateral) and the location of pads/bolsters/other points of fixation. This provides constraints to spine motion on the operating table.
Surgical instrument position—provided by navigation and robotic systems, which currently track the position of surgical instruments
   The location and trajectory of forces imparted on the spine allows the system to account for these forces in the model.
Forces imparted upon the spine-Two types of forces can be applied during surgery
   Forces delivered through the surgical field
      These include intentional forces as part of the surgery, such as the downward force of a surgical drill as well as unintentional forces, such as the retraction for exposure.
   Forces delivered outside the surgical
      This can include the force of the surgeon leaning on the patient, or a patient coughing.
   Tracking these forces, in conjunction with the position and trajectory of those forces, provides data to account for instantaneous and aggregate displacement moments on the vertebra.
Surgical interventions such as bone removal or implant placement
   Provided by the surgical team, navigation system, optical tracking or a combination, allows the model to account for intentional changes to anatomy, which lead to changes in the prediction of vertebral position.

Turning our attention back to FIG. 1, at step 104, receiving a plurality of patient-specific features (see discussion above) associated with a particular one surgical patient. Then certain operations are performed during the real-time surgery in an iterative fashion for a defined period. That is, at step 106, providing the plurality of patient-specific features received to the trained predictive model and using at least the trained predictive model, creating a segmental motion model specific to the particular one surgical patient. Initially, the training data would consist of volumetric spine images with manually defined anatomical landmarks and ground truth anthropomorphic measurement values. A variety of supervised regression loss functions can train the parameters of the neural networks to output accurate anthropomorphic measurements from the input images in an automated manner. Once automated, the anthropomorphic data can be extracted from spine images and included in the patient's biomedical profile as an input for the segmental tracking algorithm training. In an embodiment, the machine learning architecture for segmental tracking could be defined as follows:

### 1. Imaging Encoder

3D U-Net CNN encodes imaging data into a feature representation
Inputs:
   T1 and T2 MRI volumes (128×128×64 voxels)
   CT volumes (128×128×64 voxels)
   Lateral and anteroposterior x-ray images (128×128 pixels)
MRI Encoder:
   3D U-Net Architecture
   Encoder: 4 convolutional blocks down sampling
   Bottleneck: 2 convolutional blocks
   Decoder: 4 convolutional blocks up sampling
   Output: 256-dim encoding
CT Encoder:
   3D Convolutional Encoder
   Input layer
   4 convolutional blocks, max pooling
   2 fully-connected layers
   Output: 256-dim encoding
X-ray Encoder:
   2D Convolutional Encoder
   Input layer
   4 convolutional blocks
   2 fully-connected layers
   Output: 256-dim encoding*2 (for lateral and AP films)
Modality Fusion:
   256-dim MRI, CT, X-ray(1) and X-ray(2) vectors
   Concatenation layer
   Fully connected layer reduces dimensions
   Outputs fused 256-dim multimodal encoding

### 2. Tabular Encoder

Multi-layer perceptron neural network encodes non-imaging input data into feature vectors
Input Layer:
   Biomarkers: 16 normalized features
   Demographics: 16 normalized features
   Anthropometrics: 32 normalized features
   Biomechanics: 32 normalized features
Hidden Layers:
   Layer 1:128 nodes, ReLU activation
   Layer 2:64 nodes, ReLU activation
   Dropout: 0.25 after each hidden layer
Output Layer:
   64 nodes, sigmoid activation
   Outputs 64-dim feature encoding
Regularization:
   L2 kernel regularization
   Early stopping based on validation loss

### 3. Feature Concatenation

Imaging features concatenated with biomarker, demographic, anthropomorphic, and biomechanical encodings
   Imaging features (256-dim)
   Biomarkers (16-dim)
   Demographics (16-dim)
   Biomechanics (32-dim)
   Anthropomorphic (32-dim)
   Total Input: 352-dim

### 4. Spine Modeling Network

Sequence model (e.g., Long Short Term Memory or Transformer (LSTM)) takes concatenated features as input and predicts time-series spine mechanics variables:
   Intervertebral rotations
   Intervertebral translations
   Spine loads (forces and moments)
For LSTM
   3-Layer LSTM network
      Layer 1:256 LSTM units
      Layer 2:128 LSTM units
      Layer 3: Output layer
   Targets: For each of the intervertebral joints at each timepoint:
      3 rotational displacements (deg)
      3 translational displacements (mm)
      3 intersegmental forces (N)
      3 intersegmental moments (Nmm)
   Output Layer:
      For each spinal joint, outputs a 60-dim vector at each timeframe consisting of:
         9-dim rotation vector
         9-dim translation vector
         9-dim force vector
         9-dim moment vector

### 6. Loss Function:

MSE loss between predicted and Finite Element Analysis (FEA)-simulated spine kinematics and kinetics.
   The loss function facilitates model learning by providing a differentiable objective metric to optimize network weights towards superior generalization capability in spine mechanics from multimodal patient data. More particularly:
1. Purpose: The loss function defines the objective that the model parameters are optimized to minimize during training. By minimizing a loss that compares the model's predictions to known targets, the model is forced to learn to produce more accurate predictions.
2. Role in Training: During training, the loss function is used to compare each batch of predictions from the model against the known ground truth biomechanics values from finite element analysis simulations. Errors between the predicted and true values are quantified by the loss to judge current model performance.
3. Updating Parameters: The gradient of the loss function is then propagated backwards to update the trainable parameters (e.g., weights and biases) of the neural networks in the model through backpropagation and gradient descent. Parameters are tweaked to lower the loss error on training data predictions over successive iterations.
4. Evaluating Performance: Tracking validation loss determines how increasing performance on the training set transfers to unseen data. The optimized model with lowest validation loss is selected to provide the best generalizability to new patient data, enabling accurate spine biomechanics predictions.
   Finite element analysis (FEA) would be performed independently outside of the machine learning architecture to generate synthetic ground truth data for training. The steps involving FEA are:
1. Spine Modeling:
   Construct spine FEA model from MRI and anthropometry population statistics
   Define vertebrae and intervertebral disc meshes, material properties
2. Simulating Loading Conditions:
   Apply various loads, motions to model
   Solve for internal stress/strain state over time
3. Calculating Biomechanics:
   From FEA solutions, compute intersegmental rotations, translations
   Derive spinal loads-forces in x/y/z dirs.
4. Pair with Synthetic Inputs:
   Generate matched sets of synthetic imaging, biomarkers etc.
      FEA provides the target ground truth outputs that the machine learning model aims to predict from the synthetic inputs. The loss function compares network predictions to these FEA-derived targets during training. Alternatively, a Graph Neural Network approach could be taken for the segmental tracking architecture as follows:
      Using the same inputs:
1. Graph Construction
   Vertices: each vertebrae
   Features: imaging, biomarkers etc. as connected nodes
2. Graph Encoder
   Graph convolutional layers
   Learn topology embeddings
   Output 256-dim vertex embeddings
3. Graph Decoder
   Graph LSTM layers
   Message passing between vertices
   Output 60-dim biomechanics per vertex
4. Loss
   Mean squared error
   Compare to ground-truth simulations.

Turning our attention again back to FIG. 1 and consistent with the above discussion, at step 108, receiving real-time surgical data specific to the particular one surgical patient, and at step 110 (e.g., intraoperative data comprising one or more of elapsed surgical time, anesthesia administered, patient position, surgical instrument positioning data, imparted spinal force data, intraoperative imaging and surgical intervention). Then, at step 112, applying the segmental motion model updated by at least inputting the real-time surgical data collected into the segmental motion model updated and receiving from the segmental motion model updated a series of vertebral location predictions specific to movements and location of a spine of the particular one surgical patient. This step also includes updating the trained predictive model using at least the real-time surgical data specific to the particular one surgical patient received and the real-time interoperative data received and using the trained predictive model updated, updating the segmental motion model created specific to the particular one surgical patient. At step 114, providing to a surgical guidance system (e.g., a surgical navigation system and/or a surgical robotic system) at least the series of vertebral location predictions received for use in performing the real-time surgery in accordance with a surgical plan. In the subject embodiment, steps 108 through 114 are iteratively repeated for a defined period (e.g., all or a portion of the duration of the surgery) during the real-time surgery. Optionally, a surgical plan associated with the real-time surgery is updated based on at least the series of vertebral location predictions received. For example, a surgical plan may include at least performing bone removal from the spine of the particular one surgical patient or placement of one or more implants in the spine of the particular one surgical patient. At steps 116 and 118, as an option, a decision may be made to update the training dataset with the surgical patient's biomedical profile given the performance of the real-time surgery and then the operations end.

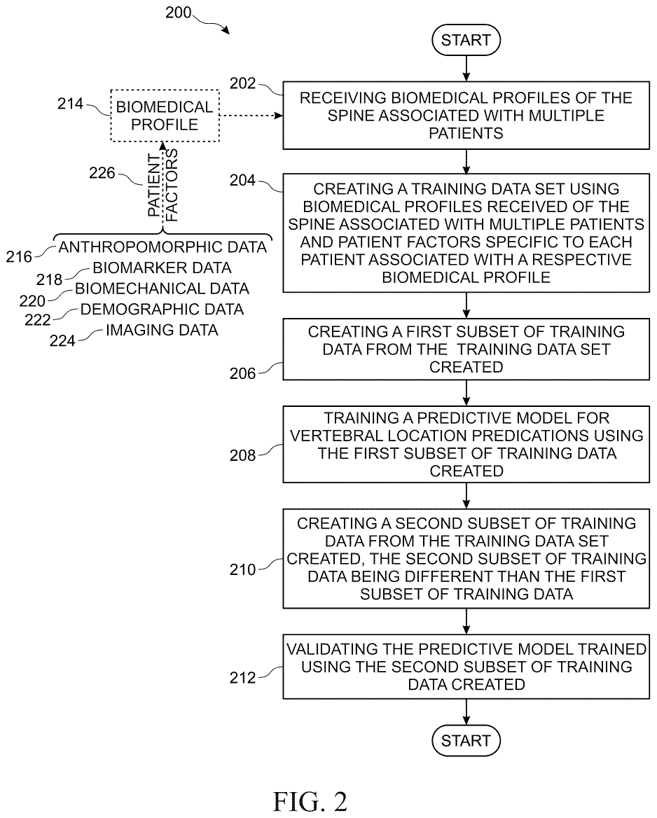

Turning our attention to FIG. 2, a flowchart of illustrative operations 200 is shown for training a predictive model useful in predicting spinal motion and vertebral locations and movement using biomedical profiles of patient spines and surgical patient-specific features in accordance with an embodiment. More particularly, at step 202, receiving biomedical profiles of the spine associated with multiple patients. As detailed above, such biomedical profiles may comprise anthropomorphic data 216, biomarker data 218, biomechanical data 220, demographic data 222, and imaging data 224. At step 204, creating a training set using the biomedical profiles 214 of the spine associated with the multiple patients and patient factors 226 specific to each patient, as also detailed hereinabove, associated with a respective biomedical profile. At step 206, creating a first subset of training data (e.g., seventy percent (70%) of the total training data available) from the created training dataset. In this way, the training operation identifies relevant patient features and how they affect spinal mechanics. Illustratively, the biomechanical profiles 214 may be obtained through cadaveric and clinical studies of spine motion under various loads and perturbations (e.g., forward bending). The differential response in patient factors that are present allows for associations to be made between spinal motion and the various patient factors. Then, at step 208, training a predictive model for vertebral location predictions using the first subset of training data created. A second subset of training data is created, at step 210, from the balance of the available training data (e.g., thirty percent (30%)) not used in creating in the first training data subset. Thus, the first subset and second subset of training data are different. At step 212, validating the trained predictive model using the second subset of training data created. The performance of the predictive model is predominantly predicated on the ability to adjust expected spinal biomechanics based on the relevant patient features and may be evaluated against actual biomechanics similar to those applied in the training phase. For example, a patient that is eighty-nine (89) years old will typically have reduced mobility of the spinal segments in all axes of motion compared to some baseline in all axes. Further, additional patient features (e.g., diabetes, body mass index (BMI) and spinal parameters, to name just a few) will increase or decrease these baseline weights based on their associations with motion, as defined during the training phase.

Thus, in accordance with the disclosed embodiments, in utilizing the predictive model in the course of navigated and/or robotic surgery, the available and relevant features of a given patient are provided to the predictive model. This includes preoperative imaging, demographics, comorbidities and biomarkers which allows the predictive model to provide predictions specific to the patient undergoing the real-time surgery. At the point of use, the model is therefore customized to that patient using their spinal geometry as well as their specific biomarkers and demographics. Bone material properties and disc stiffness values may also be tuned and fine-tuned in accordance with the patient's specific measurements. Further, any reference location on the patient (i.e., how the patient is tracked by the surgical guidance system) or the robotic docking position (e.g., spinous process) is specified to the model if such are utilized thereby informing the predictive model of the rigid position, if any, from which to provide predictions. During the surgery, the predictive model continuously receives data from the surgical guidance system as well as other sources to inform the predictive model of surgical events and interventions performed on the spine. For example, the position of surgical instruments, the forces applied to the spine or the removal of anatomy (e.g., laminectomy). In this way, these various inputs allow the predictive model to provide ongoing updates to the segmental tracking to the surgical guidance system in real-time.

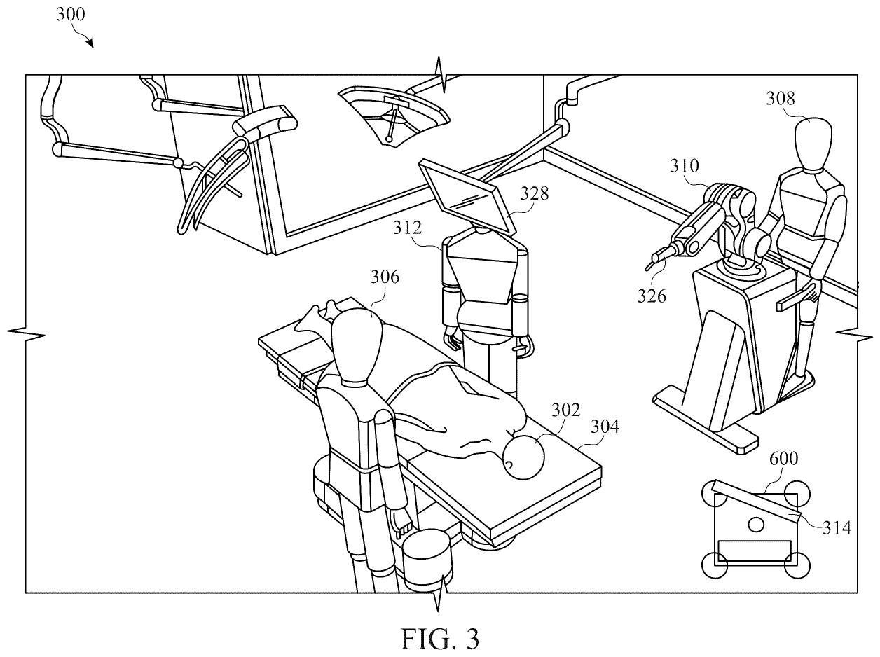

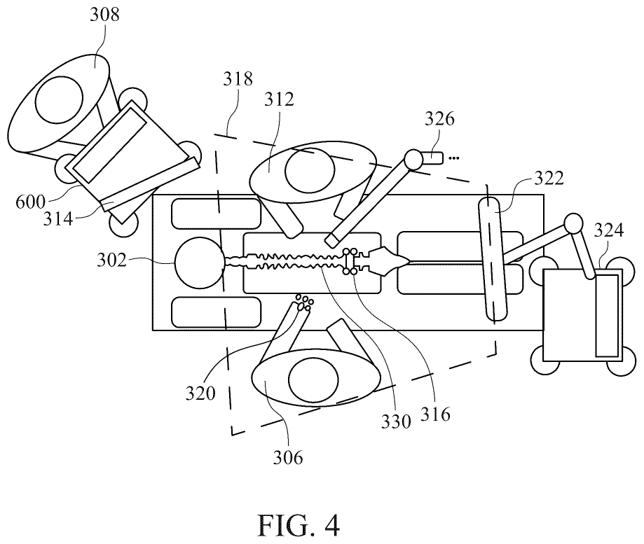

Turning our attention to FIGS. 3 and 4 which will be discussed together, FIG. 3 presents a diagram of an illustrative surgical operation room 300 in which a vertebral location prediction system, a surgical guidance system (i.e., a surgical robotic system 310) and other surgical equipment are disposed around a surgical patient and surgical personnel in accordance with an embodiment, and FIG. 4 presents a diagram of an illustrative real-time surgical operation in the surgical operation room 300 in which the vertebral location prediction system 600, the surgical robotic system 310, a surgical navigation system 324 and other surgical equipment (e.g., overhead display 328) are in use and disposed around a surgical patient 302 and surgical personnel in accordance with an embodiment. More particularly, these figures illustrate the vertebral location prediction system 600 having display 314, the surgical robotic system 310 having robotic arm 326 in the operating room 300, and the surgical navigation system 324 having display 322 and field of vision 318. Of course, there are many different configurations for the operating room 300 and the subject example is not meant to be exhaustive in any way with respect to personnel and/or equipment. Further, in some implementations only one of the surgical robotic system and the surgical navigation system may be present in further implementations they may be combined into a single system. In some implementations, one or more surgeons 306, surgical assistants 312 and/or other surgical technicians 308 are present for performing a real-time surgical operation on a patient 302 positioned on operating table 304 using the surgical robotic system 310 and/or the surgical navigation system 324. In the operating room 300, the surgeon 306 may be guided by the surgical robotic system 310 to accurately execute the operation which may be achieved by robotic guidance of the surgical tools 320, including ensuring the proper trajectory of the tool (e.g., drill or screw). In some implementations, the surgeon 306 defines the trajectory intra-operatively with little or no pre-operative planning and in other situations a surgical plan has been defined prior to the initiation of the real-time surgical procedure. The surgical robotic system 310 allows the surgeon 306 to achieve desired alignment of the tool for performing crucial steps of the surgical procedure. Operation of the robot arm 326 by the surgeon 306 (or other operator) permits movement of the tool in a measured, even manner that disregards accidental, minor movements of the surgeon 306. The surgeon 306 moves the tool holder to achieve proper trajectory of the tool (e.g., a drill or screw) prior to operation or insertion of the tool into the patient 302 (e.g., into the spine 330 of the patient 302). Once the robotic arm is in the desired position, the arm is fixed to maintain the desired trajectory. The tool holder serves as a stable, secure guide through which a tool may be moved through or slid at an accurate angle. Thus, the disclosed technology provides the surgeon with reliable instruments and techniques to successfully perform his/her surgery.

In some embodiments, the operation may be spinal decompression surgery, such as a discectomy, a foraminotomy, a laminectomy, or a spinal fusion. In some implementations, the surgical robotic system 310 and/or the surgical navigation system 324 may include a mobile cart for positioning these systems in proximity to the operating table 304 without being attached thereto, thereby providing maximum operating area and mobility to the surgeon 306 and others around the operating table 304 and reducing clutter thereon. In alternative embodiments, the surgical robotic system 310 and/or the surgical navigation system 324 may be secured directly to the operating table. In certain embodiments, both the operating table the and the mobile cart may be secured to a common base to prevent any movement of the cart or table in relation to each other. Thus, in certain embodiments, the surgical robotic system 310 and/or the surgical navigation system 324 are mobile so as to permit a user/operator, such as a technician, nurse, surgeon, or any other medical personnel in the operating room 300, to move these systems to different locations before, during, and/or after the surgical procedure. The robotic arm 326 may include a force control end-effector configured to hold a surgical tool. The robot may be configured to control and/or allow positioning and/or movement of the end-effector with at least four degrees of freedom (e.g., six degrees of freedom, three translations and three rotations) . In some implementations, the robotic arm 326 may be configured to releasably hold a surgical tool, allowing the surgical tool to be removed and replaced with a second surgical tool. The system may allow the surgical tools to be swapped without re-registration, or with automatic or semi-automatic re-registration of the position of the end-effector. In some implementations, the surgical robotic system 310 may further comprise a tracking detector (not shown) that captures the position of the patient and different components of the surgical robotic system 310, and a display screen (not shown) that displays, for example, real time patient data and/or real time surgical robot trajectories. In some implementations, the tracking detector monitors the location of the patient 304 and the robotic arm 326. The tracking detector may be a camera, a video camera, an infrared detector, and/or sensors for tracking or any other motion detecting apparatus.

In accordance with the principles of the disclosed embodiments, the vertebral location prediction system 600 is in communication with the surgeon 306 (e.g., through the overhead display 328), the surgical robotic system 310, and/or the surgical navigation system 324 to improve upon the real-time surgery operations and the overall success of the surgery. For example, the vertebral prediction calculated and supplied by the vertebral location prediction system 600 may translate into proposed trajectories for the robotic arm 326 of the surgical robotic system 310 to a patient operation site along the patient's spine 330 using patient reference array 316. By continuously monitoring the patient, the real-time surgery, navigation positions, and robotic arm positions, the vertebral location prediction system 600 may calculate updated predictive model, updated segmental motion models that will transform trajectories and visually display these transformed trajectories on the overhead display 328 and/or the display 314, for example, to inform and guide the surgeons 306 and/or the technician 308 in the operating room 300. In addition, in certain embodiments, the surgical robotic system 310, for example, may also change its position and automatically position itself based on trajectories calculated from the real-time data supplied by the vertebral location prediction system 600. For instance, the trajectory of the end-effector can be automatically adjusted in real-time to account for movement of the vertebrae or other part of the patient 302 during the surgical procedure.

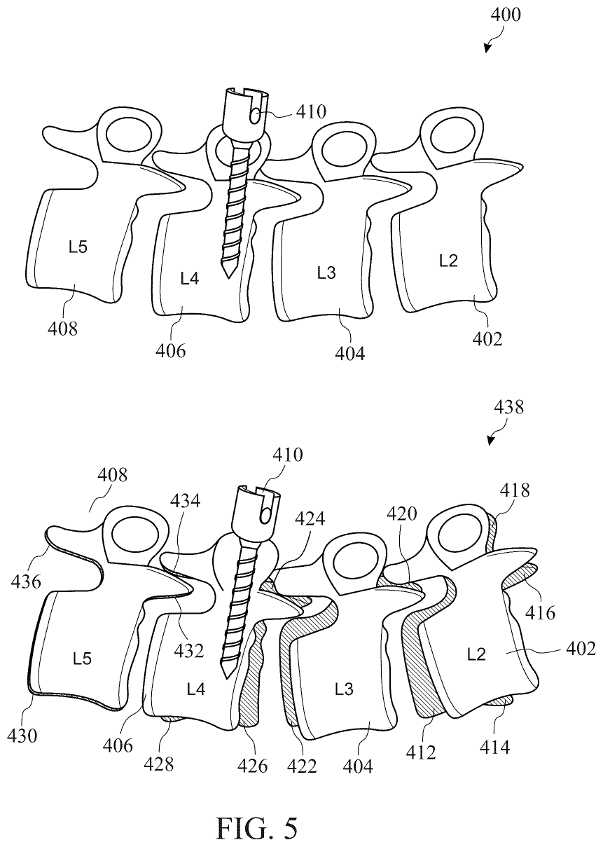

For instance, with respect to vertebrae movement, turning our attention to FIG. 5 an illustrative anatomical example 438 directed to the lumbar spine is shown based on predictive modelling and a comparison of anatomy and location thereof based on predictive modelling in accordance with an embodiment and an illustrative anatomical example 400 without the use of such predictive modelling. As will be appreciated by one skilled in the art, the lumbar spine consists of the five bones (i.e., vertebra), known as L1, L2, L3, LA, and L5, in your lower back and provides support for the weight of your body, surrounds and protects your spinal nerves, and allows for a wide range of body motions. The lumbar vertebra L1 through L5 are the largest in the entire human spine and are located below a human's twelve (12) chest (thoracic) vertebra and above the five (5) fused bones that form the triangular-shaped sacrum bone. Compared with other spine vertebrae, these lumbar vertebrae are larger, thicker and more block-like bones. The lumbar vertebrae provide stability for a person's back and spinal column and allow for a point of attachment for many muscles and ligaments. The lumbar spine and the muscles and ligaments that attach to them allow a person to walk, run, sit, lift and move their body in all directions. Thus, many conditions can affect this area of the spine, including lower back pain, spondylosis, spondylolisthesis, and stenosis, and these lumbar spine vertebrae are frequently the subject of surgical operations. The illustrative anatomical example 400 shows lumbar vertebra L2 402, L3 404, L4 406, and L5 408, respectively, that are subject to a surgical procedure involving the laminectomy of L2, L3, L4 and L5 and the insertion of implant 410 (a pedicle screw) into the lumbar vertebra L4 406. While 400 reflects the intended position of the screw 410, the actual positioning of these lumbar vertebrae during the real-time surgery can be vastly different from the expected positioning, as shown in 400, given the changes to the spine as a result of surgery (e.g., laminectomy) and the forces applied to the spine. This difference in position between expected and reality can lead to screw malposition. More particularly, the illustrative anatomical example 438 shows the actual anatomy based on using the predictive modelling employing segmental tracking of the disclose embodiments overlaid on the expected anatomy (shaded regions) without the use of such predictive modelling (i.e., illustrative anatomical example 400). As can be seen by studying this comparison, the illustrative anatomical example 438 has different vertebral projected positioning as compared the illustrative anatomical example 400, depicted by regions 412, 414, 416, 418, 420, 422, 424, 426, 428, 430, 432, 434, and 436 which were not updated using the predictive modelling employing segmental tracking of the disclose embodiments. In some cases, for example lumbar vertebra L2 402, L3 404, and L4 406, the variations between the projected positioning of the lumbar vertebrae is dramatic and these mismatches demonstrate the advantage and utility of the present invention given that projected positioning of the lumbar vertebra 406 into which the implant 410 is to be inserted can be adversely affected and cause significant morbidity if the lumbar vertebra's projected positioning is inaccurate and the surgeon's insertion the implant 410 may negatively affect adjacent anatomy. Of course, the above-described illustrative lumbar spine example is only possible application of the principles of the disclosed embodiments which apply equally to other spinal areas including, but not limited to, the cervical spine.

In this way, during navigated and/or robotic surgery, the predictive model may be advantageously used to provide segmental localization of the patient's spine thereby providing and integrating the practical application and technical benefit to, for example, surgical guidance systems such as surgical navigation systems and/or a surgical robotic systems. As detailed above, the predictive model uses various inputs from the surgical field to provide patient-specific predictions of vertebral positions. These inputs may include the location of surgical instruments, surgical interventions delivered (e.g., laminectomy), intraoperative imaging and forces/pressures applied to the patient's spine. The predictions are then provided to the navigation and/or robotic surgical system to update the expected vertebral positions of the patient in real-time. The surgeon(s) may then have a visual presentation of the “corrected” position of the spinal segments on their display.

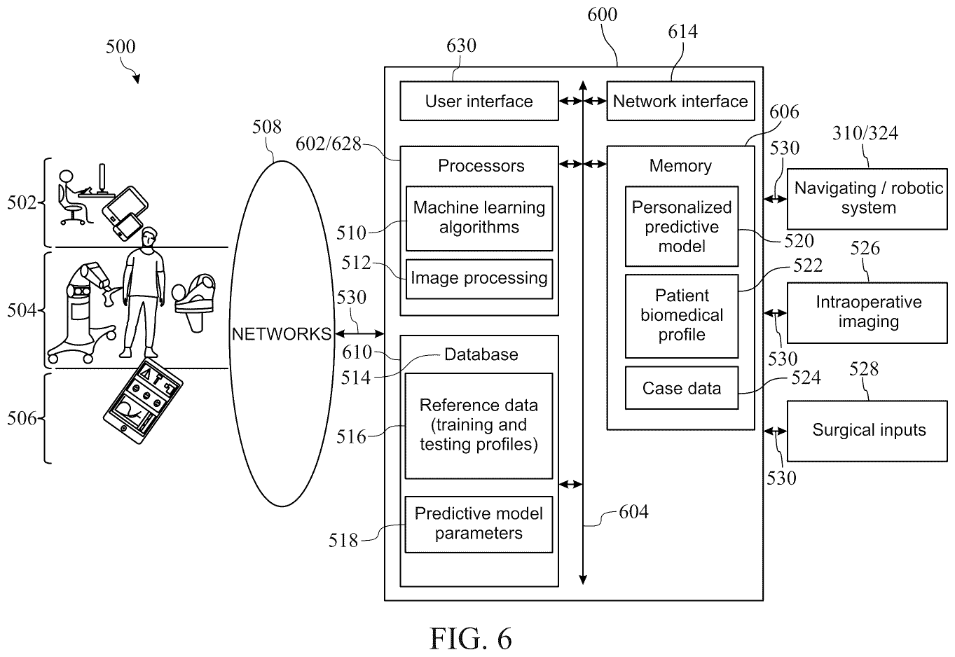

Turning our attention to FIG. 6, an illustrative navigated surgery workflow 500 using the vertebral location prediction system 600 configured in accordance with an embodiment. More particularly, the illustrative navigated work flow 500 comprises three (3) stages of workflow: a pre-operative stage 502; an intra-operative stage 504; and post-operative stage 506: During the pre-operative stage 502, a user (e.g., the surgeon 306) generates a surgical plan based on analyzed patient images with assistance from the vertebral location prediction system 600. Further, during the pre-operative stage 502 the various training data necessary for training the predictive model is collected and analyzed, as detailed herein above. During the intra-operative stage 504, the user is provided navigated assisted by the vertebral location prediction system 600 precise surgical plan execution as previously detailed hereinabove. During the post-operative stage 506, post-operative feedback data characterizing surgery outcomes is collected, for example, as detailed above an update to the training dataset with the surgical patient's biomedical profile may made. Data obtained across all phases 502-506 can be stored in a central database (not shown) for use by the vertebral location prediction system 600 to train a machine learning model. As noted above, the machine learning model can include AI processes, neural network components, etc. and may be trained over time and used to generate predictive models that allow for more accurate surgical plans and intraoperative guidance that result in improved surgical outcomes. Although FIG. 6 shows a single computer providing post-operative feedback data during the post-operative stage 506 through one or more networks 508 (e.g., public networks (Internet) and or private networks (local area network), it is to be understood that numerous network computers (e.g., servers, access points, etc.) may be used in the same fashion.

For example, the one or more networks 508 may have a cloud network services architecture connected with several data processing systems through a cloud network. As will be appreciated, a “data processing system” in the context herein may comprise a wide variety of devices such as any type of hardware device, mobile devices, smartphones, laptop computers, desktop computers, tablets, servers, and wearable devices, to name just a few, that execute applications (e.g., a mobile application) in accordance with the principles of the disclosed embodiments herein. The cloud network may include at least server(s), access point(s) and database(s). Cloud, cloud service, cloud server and cloud database are broad terms and are to be given their ordinary and customary meaning to one of ordinary skill in the art and includes, without limitation, any database, data repository or storage media which store content typically associated with and managed by a user. A cloud service may include one or more cloud servers and cloud databases that provides for the remote storage of content as hosted by a third-party service provider or operator. A cloud server may include an HTTP/HTTPS server sending and receiving messages in order to provide web-browsing interfaces to client web browsers as well as web services to send data to integrate with other interfaces (e.g., as executed on the vertebral location prediction system 600). The cloud server may be implemented in one or more servers and may send and receive content in a various forms and formats, user supplied and/or created information/content and profile/configuration data that may be transferred from or stored in a cloud database. A cloud database may include one or more physical servers, databases or storage devices as dictated by the cloud service's storage requirements. The cloud database may further include one or more well-known databases (e.g., an SQL database) or a fixed content storage system to store content, user profile information, configuration information, administration information and any other information necessary to execute the cloud service. In various embodiments, one or more networks providing computing infrastructure on behalf of one or more users may be referred to as a cloud, and resources may include, without limitation, data center resources, applications (e.g., software-as-a-service or platform-as-a-service) and management tools. The term “cloud computing” as referred to herein implies the on-demand availability of computer system resources, especially data storage and computing power, without direct active management by the user, The term is generally used to describe data centers available to many users over the Internet. Large clouds, predominant today, often have functions distributed over multiple locations from central servers. If the connection to the user is relatively close, it may be designated an edge server Clouds may be limited to a single organization (e.g., enterprise clouds), be available to many organizations (e.g., public cloud), or a combination of both (e.g., hybrid cloud). Cloud computing relies on sharing of resources to achieve coherence and economies of scale. Advocates of public and hybrid clouds note that cloud computing allows companies to avoid or minimize up-front IT infrastructure costs. Proponents also claim that cloud computing allows enterprises to get their applications up and running faster, with improved manageability and less maintenance, and that it enables IT teams to more rapidly adjust resources to meet fluctuating and unpredictable demand.

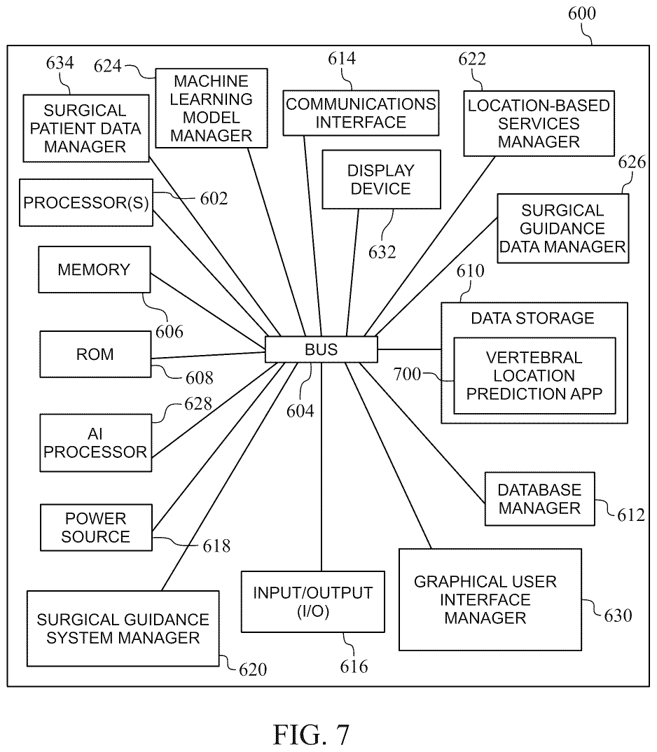

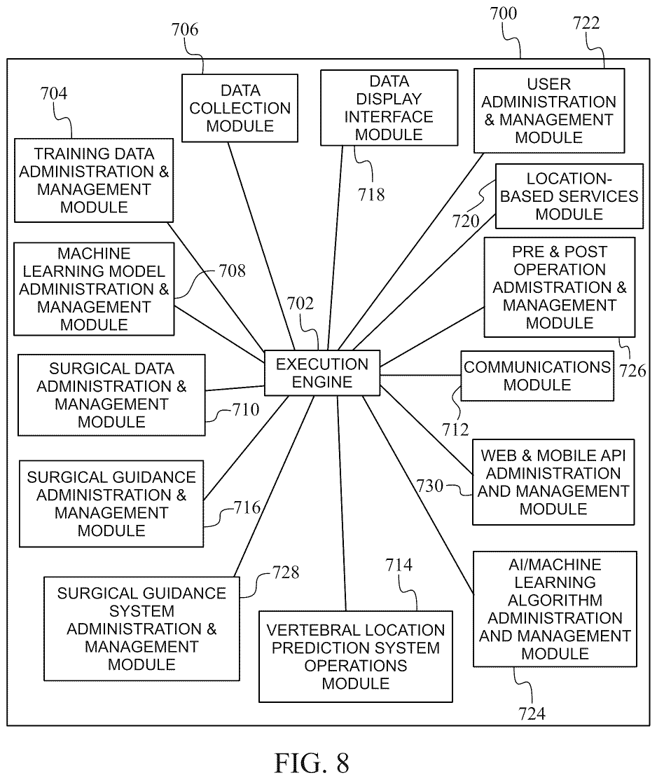

As shown in FIGS. 6 and 7 which will be discussed together, the vertebral location prediction system 600 is configured in accordance with an embodiment. As shown, the vertebral location prediction system 600 comprises processor 602 and AI processor 628 for executing program code (e.g., the vertebral location prediction app 700; see also, FIG. 8) and communications/network interface 614 for managing communications to and from the vertebral location prediction system 600 and the surgical guidance systems (e.g., the surgical navigation system 324 and the surgical robotic system 310), exchanging intraoperative imaging 526, and exchanging surgical inputs 528, as detailed previously. Memory 606, data storage 610, and/or read-only memory (ROM) 608 may be used for storing program code and data, and power source 618 for powering the vertebral location prediction system 600. The memory 606 is coupled to the bus 604 for storing computer-readable instructions (e.g., execution of the vertebral location prediction app 700) to be executed by the processor 602 (and/or AI processor 628). Processors 602 and/or 628 may execute one or more machine learning algorithms 510 and image processing 512. The processor 602 (and/or AI processor 628) may include both general and special purpose microprocessors and may be the sole processor or one of multiple processors of the device. Further, the processor 602 (and/or AI processor 628) may comprise one or more central processing units (CPUs) and may include, be supplemented by, or incorporated in, one or more application-specific integrated circuits (ASICs) and/or one or more field programmable gate arrays (FPGAs). The memory 606 may also be utilized for storing temporary variables or other intermediate information during the execution of the instructions by the processor 602 (and/or AI processor 628). The memory 606 may also store personalized predictive model 520, patient biomedical profile 522 and case data 524. The data storage device 610, such as a magnetic, optical, or solid-state device may be coupled to the bus 604 for storing information and instructions for the processor 602 (and/or AI processor 628) including, but not limited to, the vertebral location prediction app 700. For example, the data storage device 610 may store one or more database comprising reference data (training and testing profiles) 516 and predictive model parameters 518. Data storage device 610 and the memory 606 may each comprise a non-transitory computer readable storage medium and may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Surgical guidance system manager 620, surgical patient data manager 634, surgical guidance data manager 626, and database manager 612 may be used to manage the delivery and storage of content, data, and other information associated with the various operations involving interaction and communication between the vertebral location prediction system 600 and the surgical guidance systems of the disclosed embodiments as detailed above. Machine learning model manager 624 manages the predictive modelling and segmental motion model operations as previously detailed hereinabove. Location-based services manager 622 facilitates the delivery of location-based services (e.g., Global Position System (GPS) tracking) thereby allowing the vertebral location prediction system 600 to register the exact location of users of the vertebral location prediction system 600, for example, as the user roams from one location to another location (e.g., operating room to operating room) such that the services offered via the predictive modelling using segmental tracking processing hereunder may be tailored to a current location and/or the needs of the user as they may change based on their current location which may influence their selection of surgical equipment or surgical personnel, for example.

The input/output devices 616 may include peripherals, such as a biometric reader, an NFC device (e.g., NFC tag reader), camera, printer, scanner (e.g., a QR-code scanner), touchscreen display, etc. For example, the input/output devices 616 may include a display device such as a cathode ray tube (CRT), plasma monitor, liquid crystal display (LCD) monitor or organic light-emitting diode (OLED) monitor for displaying information to the user, a keyboard, and a pointing device such as a mouse or a trackball by which the user can provide input to the vertebral location prediction system 600 or an associated display device 632, for example, that may also be managed by graphical user interface generator 630. The communications/network interface 614 is used to facilitate communications across various types of communications links (e.g., communications links 530) within the one or more networks 508 architecture, for example. This may take the form, for example, of a wide area network connection that communicatively couples the vertebral location prediction system 600 with the access points of the one or more networks 508 which may be a cellular communications service. Similarly, communications managed by the communications/network interface 614 may take the form, for example, of a local Wi-Fi network interface or Ethernet interface the communicatively couples the vertebral location prediction system 600 with the well-known Internet, a LAN, and ultimately the vertebral location prediction system 600. In the instant embodiment, the vertebral location prediction app 700 and/or the communications/network interface 614 may include a communications stack for facilitating communications over the respective communications link. The vertebral location prediction system 600 may also communicate with other devices via a network (e.g., a wireless communications network) or communications protocol (e.g., Bluetooth®). For example, such communications interfaces may be a receiver, transceiver, or modem for exchanging wired or wireless communications in any number of well-known fashions. For example, the communications/network interface 614 may be an integrated service digital network (ISDN) card or modem/router used to facilitate data communications of various well-known types and formats. Further, illustratively, the communications/network interface 614 may be a LAN card used to provide data communication connectivity to a comparable LAN. As will be appreciated, the functionality of the communications/network interface 614 is to send and receive a variety of signals (e.g., electrical, optical, or other signals) that transmit data streams representing various data types.

Turning our attention to FIG. 8, an illustrative architecture for the operation of the vertebral location prediction app 700 is presented in accordance with an embodiment. As will be appreciated, the architecture may be used, illustratively, in conjunction with the vertebral location prediction system 600 or any data processing system for launching and executing the vertebral location prediction app 700 and its associated operations. As shown, the architecture for the operations of the vertebral location prediction app 700 provides several interfaces and engines used to perform a variety of functions such as the collection, aggregation, manipulation, processing, analyzing, verification, authentication, and display of applicable real-time information and data that are useful to realize the delivery of the operations for predicting spinal motion and vertebral locations and movement in real-time surgeries using predictive models generated using biomedical profiles of patient spines and surgical patient-specific features in accordance with the disclosed embodiments. More particularly, data display interface module 718 and communications module 712 are used to facilitate the input/output and display of electronic data and other information to, illustratively, the users (e.g., the surgeon 306) employing the vertebral location prediction system 600 (e.g., a touch screen of the vertebral location prediction system 600) and executing the vertebral location prediction app 700. The data collection module 706 facilitates data gathering from the plurality of users and other third parties. The location-based services module 720 provides for the delivery of location-based services in order for the geographic locations of the users to be identified and displayed (e.g., GPS locations). The communications module 712 will also facilitate communications by and through the vertebral location prediction system 600, for example.

Execution engine 702 may be employed to deliver the operations for predicting spinal motion and vertebral locations and movement in real-time surgeries using predictive models herein through the execution of the vertebral location prediction app 700. In such delivery, the execution engine 702 will operate and execute, as further detailed herein below, with at least the following program modules: training data administration and management module 704, data collection module 706, machine learning administration and management module 708, surgical data administration and management module 710, communications module 712, vertebral location prediction system operations module 714, surgical guidance administration and management module 716, user administration and management module 722, AI/machine learning algorithm administration and management module 724, pre and post operation administration and management module 726, surgical guidance system administration and management module 728, and web and mobile API administration and management module 730. Further, in an embodiment, data display interface module 718 may include a graphical user interface module, and the communications module 712 are used to facilitate the input/output and display of electronic data and other information (e.g., a graphical user interface) to, illustratively, the users employing the vertebral location prediction system 600 (e.g., a touch screen) and executing the vertebral location prediction app 700. The data collection module 706 facilitates various types of data collection from the plurality of users and systems involved in the predictive modelling operations of the disclosed embodiments. The operations executed by each and every of the foregoing modules are, for example, as discussed throughout this disclosure.

Those skilled in the art will appreciate that the present disclosure contemplates the use of systems configurations and/or computer instructions that may perform any or all of the operations involved in the predictive modelling using segmental motion and tracking operations herein. The disclosure of computer instructions that include, for example, the vertebral location prediction app 700 and the vertebral location prediction system 600 instructions is not meant to be limiting in any way. Those skilled in the art will readily appreciate that stored computer instructions and/or systems configurations may be configured in any way while still accomplishing the various goals, features, and advantages according to the present disclosure. The terms “program,” “application,” “software application,” and the like as used herein, are defined as a sequence of instructions designed for execution on a computer system. A “program,” “computer program,” “application,” or “software application” may include a subroutine, a function, a procedure, an object method, an object implementation, an executable application, an applet, a servlet, a source code, an object code, a shared library/dynamic load library, and/or other sequence of instructions designed for execution on a computer system. Accordingly, the applications herein may be written using any number of programming languages and/or executed on compatible platforms that will be well understood by those skilled in the art. Computer readable program instructions for carrying out operations of the disclosed embodiments may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on one or more standalone computers, partly on one or more standalone computers, as a stand-alone software package, partly on one or more standalone computers and partly on one or more remote computers, partly on one or more standalone computers and partly on one or more distributed computing environments (such as a cloud environment), partly on one or more remote computers and partly on one or more distributed computing environments, entirely on one or more remote computers or servers, or entirely on one or more distributed computing environments. Standalone computers, remote computers, and distributed computing environments may be connected to each other through any type of network or combination of networks, including local area networks (LANs), wide area networks (WANs), through the Internet (e.g., using an Internet Service Provider), or the connection may be made to external computers.

Devices or system modules that are in at least general communication with each other need not be in continuous communication with each other, unless expressly specified otherwise. In addition, devices or system modules that are in at least general communication with each other may com-municate directly or indirectly through one or more intermediaries. Moreover, it is understood that any system components described or named in any embodiment or claimed herein may be grouped or sub-grouped (and accordingly implicitly renamed) in any combination or sub-combination as those skilled in the art can imagine as suitable for the particular application, and still be within the scope and spirit of the claimed embodiments of the present invention. For an example of what this means, if the invention was a controller of a motor and a valve and the embodiments and claims articulated those components as being separately grouped and connected, applying the foregoing would mean that such an invention and claims would also implicitly cover the valve being grouped inside the motor and the controller being a remote controller with no direct physical connection to the motor or internalized valve, as such the claimed invention is contemplated to cover all ways of grouping and/or adding of intermediate components or systems that still substantially achieve the intended result of the invention. A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Those of skill in the art will appreciate that where appropriate, some embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Where appropriate, embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices. “Software” may refer to prescribed rules to operate a computer. Examples of software may include code segments in one or more computer-readable languages; graphical and or/textual instructions; applets; pre-compiled code; interpreted code; compiled code; and computer programs. A network is a collection of links and nodes (e.g., multiple computers and/or other devices connected together) arranged so that information may be passed from one part of the network to another over multiple links and through various nodes. Examples of networks include the Internet, the public switched telephone network, wireless communications networks, computer networks (e.g., an intranet, an extranet, a local-area network, or a wide-area network), wired networks, and wireless networks.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special-purpose computer, or other programmable data processing apparatus to produce a machine, such that the instructions, which execute via the processor of the computer or other programmable data processing apparatus, create means for implementing the functions/acts specified in the flowchart and/or block diagram block or blocks. The flowchart and block diagrams in the figures illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block dia-grams and/or flowchart illustration, can be implemented by special purpose hardware-based systems that perform the specified functions or acts, or combinations of special purpose hardware and computer instructions.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks. Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

It will be readily apparent that the various methods and algorithms described herein may be implemented by, e.g., appropriately programmed general purpose computers and computing devices. Typically, a processor (e.g., a microprocessor) will receive instructions from a memory or like device, and execute those instructions, thereby performing a process defined by those instructions. Further, programs that implement such methods and algorithms may be stored and transmitted using a variety of known media. When a single device or article is described herein, it will be readily apparent that more than one device/article (whether or not they cooperate) may be used in place of single device/article. Similarly, where more than one device or article is described herein (whether or not they cooperate), it will be readily apparent that a single device/article may be used in place of the more than one device or article. The functionality and/or the features of a device may be alternatively embodied by one or more other devices which are not explicitly described as having such functionality/features. Thus, other embodiments of the present invention need not include the device itself.

The term “computer-readable medium” as used herein refers to any medium that participates in providing data (e.g., instructions) which may be read by a computer, a processor or a like device. Such a medium may take many forms, including but not limited to, non-transitory, non-volatile media, volatile media, and transmission media. Non-volatile media include, for example, optical or magnetic disks and other persistent memory. Volatile media include dynamic random-access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, a RAM, a PROM, an EPROM, a FLASH-EEPROM, removable media, flash memory, a “memory stick”, any other memory chip or cartridge, a carrier wave as described hereinafter, or any other medium from which a computer can read. Various forms of computer readable media may be involved in carrying sequences of instructions to a processor. For example, sequences of instruction may be delivered from RAM to a processor, may be carried over a wireless transmission medium, and/or may be formatted according to numerous formats, standards or protocols, such as Bluetooth®, 4G, 5G, etc.

Where databases are described, it will be understood by one of ordinary skill in the art that alternative database structures to those described may be readily employed, and other memory structures besides databases may be readily employed. Any schematic illustrations and accompanying descriptions of any sample databases presented herein are exemplary arrangements for stored representations of information. Any number of other arrangements may be employed besides those suggested by the tables shown. Similarly, any illustrated entries of the databases represent exemplary information only; those skilled in the art will understand that the number and content of the entries can be different from those illustrated herein. Further, despite any depiction of the databases as tables, an object-based model could be used to store and manipulate the data types of the present invention and likewise, object methods or behaviors can be used to implement the processes of the present invention.

A “computer system” may refer to a system having one or more computers, where each computer may include a non-transitory computer-readable medium embodying software to operate the computer or one or more of its components. Examples of a computer system may include: a distributed computer system for processing information via computer systems linked by a network; two or more computer systems connected together via a network for transmitting and/or receiving information between the computer systems; a computer system including two or more processors within a single computer; and one or more apparatuses and/or one or more systems that may accept data, may process data in accordance with one or more stored software programs, may generate results, and typically may include input, output, storage, arithmetic, logic, and control units. A “network” may refer to a number of computers and associated devices that may be connected by communication facilities. A network may involve permanent connections such as cables or temporary connections such as those made through the telephone or other communication links. A network may further include hard-wired connections (e.g., coaxial cable, twisted pair, optical fiber, waveguides, etc.) and/or wireless connections (e.g., radio frequency waveforms, free space optical waveforms, acoustic waveforms, etc.). Examples of a network may include: an internet, such as the Internet; an intranet; a LAN; a wide area network (WAN), and a combination of networks, such as an internet and an intranet.

As noted above, in some embodiments the method or methods described above may be executed or carried out by a computing system including a non-transitory computer-readable storage medium, also described herein as a storage machine, that holds machine-readable instructions executable by a logic machine (i.e., a processor or programmable control device) to provide, implement, perform, and/or enact the above described methods, processes and/or tasks. When such methods and processes are implemented, the state of the storage machine may be changed to hold different data. For example, the storage machine may include memory devices such as various hard disk drives, CD, or DVD devices. The logic machine may execute machine-readable instructions via one or more physical information and/or logic processing devices. For example, the logic machine may be configured to execute instructions to perform tasks for a computer program. The logic machine may include one or more processors to execute the machine-readable instructions. The computing system may include a display subsystem to display a GUI, or any visual element of the methods or processes described above. For example, the display subsystem, storage machine, and logic machine may be integrated such that the above method may be executed while visual elements of the disclosed system and/or method are displayed on a display screen for user consumption. The computing system may include an input subsystem that receives user input. The input subsystem may be configured to connect to and receive input from devices such as a mouse, keyboard, or gaming controller. For example, a user input may indicate a request that certain task is to be executed by the computing system, such as requesting the computing system to display any of the above-described information or requesting that the user input updates or modifies existing stored information for processing. A communication subsystem may allow the methods described above to be executed or provided over a computer network. For example, the communication subsystem may be configured to enable the computing system to communicate with a plurality of personal computing devices. The communication subsystem may include wired and/or wireless communication devices to facilitate networked communication. The described methods or processes may be executed, provided, or implemented for a user or one or more computing devices via a computer-program product such as via an application programming interface (API).

Thus, the steps of the disclosed method(s) and the associated discussion herein above can be defined by the computer program instructions stored in a memory and/or data storage device and controlled by a processor executing the computer program instructions. Accordingly, by executing the computer program instructions, the processor executes an algorithm defined by the disclosed method. For example, the computer program instructions can be implemented as computer executable code programmed by one skilled in the art to perform the illustrative operations defined by the disclosed methods. Further, it will be appreciated that any flowcharts, flow diagrams, state transition diagrams, pseudo code, program code and the like represent various processes which may be substantially represented in computer readable medium and so executed by a computer, machine, or processor, whether or not such computer, machine or processor is explicitly shown. One skilled in the art will recognize that an implementation of an actual computer or computer system may have other structures and may contain other components as well, and that a high-level representation of some of the components of such a computer is for illustrative purposes.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalents.

1. A vertebral location prediction system comprising:
a processor;
a memory storing instructions that when executed cause the processor to perform operations comprising:
receiving a trained predictive model;
receiving a plurality of patient-specific features associated with a particular one surgical patient; and
during a real-time surgery involving the particular one surgical patient:
(i) providing the plurality of patient-specific features received to the trained predictive model and using at least the trained predictive model, creating a segmental motion model specific to the particular one surgical patient;
(ii) receiving real-time surgical data specific to the particular one surgical patient;
(iii) receiving real-time intraoperative data comprising one or more of elapsed surgical time, anesthesia administered, patient position, surgical instrument positioning data, imparted spinal force data, and surgical intervention;
(iv) updating the trained predictive model using at least the real-time surgical data specific to the particular one surgical patient received and the real-time interoperative data received and using the trained predictive model updated, updating the segmental motion model created specific to the particular one surgical patient;
(v) applying the segmental motion model updated by at least inputting the real-time surgical data collected into the segmental motion model updated and receiving from the segmental motion model updated a series of vertebral location predictions specific to movements and location of a spine of the particular one surgical patient;
(vi) providing to a surgical guidance vertebral location prediction system at least the series of vertebral location predictions received for use in performing the real-time surgery; and
(vii) repeating steps (ii) through (vi) for a defined period during the real-time surgery.

2. The vertebral location prediction system of claim 1, wherein the operations performed by the processor further comprise:
training a predictive model using a first training dataset comprising a plurality of biomedical spine profiles wherein each respective biomedical spine profile is associated with a respective one patient of a plurality of patients and a plurality of patient factors specific to the respective one patient.

3. The vertebral location prediction system of claim 2, wherein the operations performed by the processor further comprise:
updating the first training dataset with the particular one surgical patient's biomedical spine profile.

4. The vertebral location prediction system of claim 1, wherein the surgical guidance vertebral location prediction system is a surgical navigation vertebral location prediction system.

5. The vertebral location prediction system of claim 1, wherein the surgical guidance vertebral location prediction system is a surgical robotic vertebral location prediction system.

6. The vertebral location prediction system of claim 1, wherein the surgical guidance vertebral location prediction system comprises both a surgical navigation vertebral location prediction system and a surgical robotic vertebral location prediction system.

7. The vertebral location prediction system of claim 2, wherein each biomedical spine profile comprises imaging data and biomechanical data.

8. The vertebral location prediction system of claim 7, wherein each biomedical spine profile further comprises at least one of biomarker data, demographic data, and anthropomorphic data.

9. The vertebral location prediction system of claim 8, wherein the imaging data further comprises at least one or more spine images; the biomarker data further comprises at least one of inflammation data, bone turnover data, cartilage turnover data, and bone metabolism data; the demographic data further comprises at least one of age, sex, race, height, and weight; the anthropomorphic data further comprises one or more measurements of a body and an associated spine defined by at least one of vertebral body shape and geometry, spinopelvic parameters, spinal alignment, bone quality, intervertebral disc height and geometry, musculature parameters, ligament measurements, and body composition; and the biomechanical data further comprises at least quantitative dynamic or static measurements of spine loading or motion.

10. The vertebral location prediction system of claim 9, wherein the operations performed by the processor further comprise:
receiving a plurality of volumetric spine images;
defining a second training dataset from the plurality of volumetric spine images;
training a machine learning algorithm using the second training dataset defined; and
using the machine learning algorithm trained, extracting the anthropomorphic data from the plurality of volumetric spine images received.

11. The vertebral location prediction system of claim 1, wherein the series of vertebral location predictions specific to movements and location of a spine of the particular one surgical patient are continually updated throughout the real-time surgery.

12. The vertebral location prediction system of claim 2, wherein the training of the predictive model employs at least one image-based artificial intelligence algorithm.

13. The vertebral location prediction system of claim 12, wherein the at least one image-based artificial intelligence algorithm is a convolutional neural network (CNN) algorithm.

14. The vertebral location prediction system of claim 8, wherein the operations performed by the processor further comprise:
applying at least one statistical learning algorithm to at least the biomechanical data and the biomarker data and identifying one or more relationships therebetween that influence spine motion.

15. The vertebral location prediction system of claim 1, wherein the operations performed by the processor further comprise:
updating a surgical plan associated with the real-time surgery based on at least the series of vertebral location predictions received.

16. The vertebral location prediction system of claim 15, wherein the surgical plan includes at least performing bone removal from the spine of the particular one surgical patient or placement of one or more implants in the spine of the particular one surgical patient.

17. A vertebral location prediction system comprising:
a processor;
a memory storing instructions that when executed cause the processor to perform operations comprising:
training a predictive model using a training dataset comprising a plurality of biomedical spine profiles wherein each respective biomedical spine profile is associated with a respective one patient of a plurality of patients and a plurality of patient factors specific to the respective one patient, and wherein each biomedical spine profile comprises imaging data and biomechanical data;
receiving a plurality of patient-specific features associated with a particular one surgical patient; and
during a real-time surgery involving the particular one surgical patient:
(i) providing the plurality of patient-specific features received to the trained predictive model and using at least the trained predictive model, creating a segmental motion model specific to the particular one surgical patient;
(ii) receiving real-time surgical data specific to the particular one surgical patient;
(iii) receiving real-time intraoperative data comprising one or more of elapsed surgical time, anesthesia administered, patient position, surgical instrument positioning data, imparted spinal force data, and surgical intervention;
(iv) updating the trained predictive model using at least the real-time surgical data specific to the particular one surgical patient received and the real-time interoperative data received and using the trained predictive model updated, updating the segmental motion model created specific to the particular one surgical patient;
(v) applying the segmental motion model updated by at least inputting the real-time surgical data collected into the segmental motion model updated and receiving from the segmental motion model updated a series of vertebral location predictions specific to movements and location of a spine of the particular one surgical patient;
(vi) providing to a surgical guidance vertebral location prediction system at least the series of vertebral location predictions received for use in performing the real-time surgery, and wherein the surgical guidance vertebral location prediction system is one of a surgical navigation vertebral location prediction system alone, a surgical robotic vertebral location prediction system alone or a combination of a surgical navigation vertebral location prediction system and a surgical robotic vertebral location prediction system;
(vii) repeating steps (ii) through (vi) for a defined period during the real-time surgery.

18. The vertebral location prediction system of claim 17, wherein the operations performed by the processor further comprise:
updating a surgical plan associated with the real-time surgery based on at least the series of vertebral location predictions received, and wherein the surgical plan comprises at least performing bone removal from the spine of the particular one surgical patient or placement of one or more implants in the spine of the particular one surgical patient.

19. The vertebral location prediction system of claim 18, wherein each biomedical spine profile further comprises at least biomarker data, demographic data, and anthropomorphic data.

20. A vertebral location predication prediction system comprising:
a processor;
a memory storing instructions that when executed cause the processor to perform operations comprising:
training a predictive model using a training dataset comprising a plurality of biomedical spine profiles wherein each respective biomedical spine profile is associated with a respective one patient of a plurality of patients and a plurality of patient factors specific to the respective one patient, and wherein each biomedical spine profile comprises imaging data and biomechanical data, and at least one of biomarker data, demographic data, and anthropomorphic data;
receiving a plurality of patient-specific features associated with a particular one surgical patient; and
during a real-time surgery involving the particular one surgical patient:
(i) providing the plurality of patient-specific features received to the trained predictive model and using at least the trained predictive model, creating a segmental motion model specific to the particular one surgical patient;
(ii) receiving real-time surgical data specific to the particular one surgical patient;
(iii) receiving real-time intraoperative data comprising one or more of elapsed surgical time, anesthesia administered, patient position, surgical instrument positioning data, imparted spinal force data, and surgical intervention;
(iv) updating the trained predictive model using at least the real-time surgical data specific to the particular one surgical patient received and the real-time interoperative data received and using the trained predictive model updated, updating the segmental motion model created specific to the particular one surgical patient;
(v) applying the segmental motion model updated by at least inputting the real-time surgical data collected into the segmental motion model updated and receiving from the segmental motion model updated a series of vertebral location predictions specific to movements and location of a spine of the particular one surgical patient;
(vi) providing to a surgical guidance vertebral location prediction system at least the series of vertebral location predictions received for use in performing the real-time surgery, and wherein the surgical guidance vertebral location prediction system is one of a surgical navigation vertebral location prediction system alone, a surgical robotic vertebral location prediction system alone or a combination of a surgical navigation vertebral location prediction system and a surgical robotic vertebral location prediction system;
(vii) updating a surgical plan associated with the real-time surgery based on at least the series of vertebral location predictions received; and
repeating steps (ii) through (vii) for a defined period during the real-time surgery.
